(19) United States
(12) Patent Application Publication
(10) Pub. No.: US 2005/0287661 A1
(43) Pub. Date: Dec. 29, 2005

(54) Methods and systems for multiplexing ir-mediated heating on a microchip

(76) Inventor: James Landers, CHARLOTTESVILLE, VA (US)

Correspondence Address: BLANK ROME LLP, 600 NEW HAMPSHIRE AVENUE, N.W., WASHINGTON, DC, 20037, US

(21) Appl. No.: 10/530,728

(22) PCT Filed: Oct. 8, 2003
(86) PCT No.: PCT/US03/31806

§ 371 Date: Apr. 8, 2005

Related U.S. Application Data
(60) Provisional application No. 60/416,927, filed on Oct. 8, 2002.

(51) Int. Cl. C12M 1/34

(52) U.S. Cl. 435/303.1; 435/91.2; 435/288.4

(57) ABSTRACT

The present invention relates to methods and systems for rapid multiplexed heating of a plurality of small volume samples on a microchip. More specifically, the present invention relates to methods and systems for non-contact temperature cycling of the samples using infrared (IR)-mediated heating of small, micro to nanoliter, volume samples, wherein each cycle can be completed in as little as a few seconds. Depending on the system used, the present invention involves a spinning microchip or an immobile microchip having a plurality of micro-heating areas thereon. In the case of the spinning chip, the micro-heating areas are located in a circular configuration on the chip, so the micro-heating areas can be accessed by static heating source(s) by spinning the microchip. In case of the immobile microchip, fiber optics are used to direct radiation from a heating source or multiple heating sources directly to the micro-heating areas on a microchip.

### FIELD OF THE INVENTION

The present invention relates to methods and systems for rapid multiplexed heating of a plurality of small volume samples on a microchip. More specifically, the present invention relates to methods and systems for non-contact temperature cycling of the samples using infrared (IR)-mediated heating of small, micro to nanoliter, volume samples, wherein each cycle can be completed in as little as a few seconds.

### BACKGROUND OF THE INVENTION

There is an on-going need to miniaturize and multiplex thermocycling, especially for the polymerase chain reaction (PCR) amplification, process into a platform that is fast, convenient and inexpensive. Microtiter plate formats have been the main contributors to high throughput PCR but still utilize conventional block heater, or forced air thermocyclers. While the number of samples that can be cycled simultaneously (96, 384 or 1536) is impressive, amplification speed is not. The limitations associated with conventional thermocyclers in the past, primarily that rate at which the temperature can be changed, provides amplification times that are not as rapid as they could be. Consequently, amplifications on the order of an hour or more are still common.

Numerous analytical methods require that a sample be heated to a particular temperature and then cooled to a particular temperature. Often, sequential heating and cooling steps, known as thermocycling, are required. Various methods involve cycling through two or more stages all with different temperatures, and/or involve maintaining the sample at a particular temperature stage for a given period of time before moving to the next stage. Accordingly, thermocycling of samples can become a time consuming process. In addition, these methods often require the precise control of temperature at each stage of the cycle; exceeding a desired temperature can lead to inaccurate results.

Two factors that are typically important, therefore, in the performance of effective thermocycling on a sample are the speed and homogeneity of the apparatus and the methods used. Cycle times are largely defined by how quickly the temperature of the sample can be changed, and relate to the heat source itself and the rate of heat transfer to the sample. Uniformity of sample temperature is important to ensure that reproducible and reliable results are obtained. Typically, increasing cycle speeds makes it harder to maintain homogenous sample temperatures.

The concept of using elevated temperatures to effect chemical, biological and biochemical reactions is commonly known and expressed as the law of Arrhenius. Generally, an increase in temperature of a reaction translates into an increase in the rate of the reaction. Reaction parameters, such as the activation of the reaction, the increase in dissolution of the reaction components, the desolvation of the substrate and the specificity of the catalysis are temperature dependent. Exact or nearly exact maintenance of a reaction temperature is often critical in most biochemical/biological processes to guarantee their successful completion. Therefore, great efforts are made in the daily routine of a chemical/biochemical laboratory to control the temperature conditions during a reaction. It is expected that better temperature control increases the performance of most reactions, for example, increasing the specificity of proteolytic reactions.

There is particular interest in rapid and homogenous thermocycling when performing DNA amplification via polymerase chain reaction (PCR). PCR is a process by which a single molecule of DNA (or RNA) from an organism can be amplified by a factor of 10⁶ to 10⁹. This procedure requires the repetition of heating and cooling cycles in the presence of an original DNA target molecule, specific DNA primers, deoxynucleotide triphosphates, and DNA polymerase enzymes and cofactors. Heating accounts for a denaturing of the sample while cooling results in annealing of the sample. At a temperature typically between the denaturing and annealing temperatures, extension of the annealed primers using an enzyme occurs to replicate the DNA strand or portion of the strand. Extension of the primer can also occur at the same temperature as annealing, depending on the specifics of the reaction. Each heating/cooling cycle produces a doubling of the target DNA sequence, leading to an exponential accumulation of the target sequence. PCR based technology has been applied to a variety of analyses, including environmental and industrial contaminant identification, medical and forensic diagnostics, and biological research.

There are a number of biochemical reactions that require accurate and rapid thermocycling. Additionally, there are reactions whose specificity can be enhanced when conducted in a rapid and accurate thermocycling environment. The PCR reaction places very high demands on the accuracy of the thermocycling parameters and is, therefore, an ideal assay to test the accuracy of the thermocycling method and apparatus.

U.S. Pat. No. 4,683,202 generally describes the PCR concept, in which a stretch of DNA is copied using a polymerase. Generally, the procedure involves annealing a piece of primer DNA at a first temperature to any stretch of single-stranded DNA template with a complementary sequence. The DNA polymerase copies the primed piece of DNA at a second given temperature. At a third given temperature, the newly copied DNA and the primer dissociate from the template DNA, thereby regenerating single-stranded DNA. The temperature of the sample is returned to the first temperature to allow the primer to attach itself to any strand of single-stranded DNA with a complementary sequence, including the DNA strands that were synthesized in the immediately preceding cycle. In that manner, the template DNA is amplified or reproduced any number of times, depending on how many times the template DNA occurs in the sample, and the number of cycles completed. The procedure can also be performed using RNA.

Most existing methods and techniques of thermocycling in benchtop instrumentation are indirect with respect to the effect of the heating source on the sample. Most thermocycling approaches heat and/or cool a circulating medium, such as water or air, that affects the container which holds the sample and, subsequently, subjects the sample itself to the desired thermocycling process. The rate of the cycling process depends on the effectiveness of the heat transfer between the circulating medium and the sample.

For example, U.S. Pat. No. 5,504,007 discloses a thermocycle apparatus having a body containing a thermally conductive liquid. The liquid is contained within the body of the apparatus, and the temperature of the liquid alternated between lower and higher temperatures in repeating cycles. A well or container for holding a sample of material is held in contact with the liquid and conducts the cyclic temperature changes of the liquid to the sample.

U.S. Pat. No. 5,576,218 discloses a method for the thermocycling of nucleic acid assays using a blended fluid stream produced from constant velocity, constant volume, and constant temperature fluid streams. Using these streams, a variable temperature, constant velocity, constant volume fluid stream is introduced into a sample chamber for heating and cooling the samples contained therein. The temperature of the blended fluid stream is varied by diverting and altering the ratio of the constant temperature fluid streams relative to one another.

U.S. Pat. No. 5,508,197 discloses a thermocycling system based on the circulation of temperature controlled water directly to the underside of a thin-walled polycarbonate microtiter plate. The water flow is selected from a manifold fed by pumps from heated reservoirs.

Other methods are reported for heating a sample through the use of heated air. U.S. Pat. No. 5,187,084 discloses an apparatus and method for performing thermocycling on a sample using an array of sample containing vessels supported in a reaction chamber, through which air at controlled temperatures is forcibly circulated as a heat-transfer medium in heat exchange relationship with the vessels. The temperature of the air is controlled as a function of time to provide a preselectable sequence defining a temperature profile. The profile is a repetitive cycle that is reproduced to effect replication of and amplification of the desired sequence of the DNA.

U.S. Pat. No. 5,460,780 discloses a device for rapidly heating and cooling a reaction vessel through various temperatures in PCR amplification utilizing a device for heating at least one side wall of a reaction vessel, device for cooling the heating device at repeated intervals and device for moving the reaction vessel and/or heating and cooling relative to each other. In one embodiment, heated air is used to heat the reaction vessel.

Similarly, U.S. Pat. No. 5,455,175 demonstrates that rapid, non-contact PCR can be accomplished in glass capillaries using air heated by foam lining the chamber in which the capillaries are placed; the foam is heated first by a halogen lamp.

Another common approach for thermocycling is through intimate contact between a reaction vessel holding the reaction medium and a heating block that is rapidly heated and cooled (for example, by using a Peltier element that can both heat and cool). That is the basis of most commercially available PCR instrumentation.

For example, U.S. Pat. No. 5,525,300 discloses an apparatus for generating a temperature gradient across a heat conducting block.

U.S. Pat. No. 5,498,392 discloses chip-like devices for amplifying a preselected polynucleotide in a sample by conducting a polynucleotide polymerization reaction. The devices comprise a substrate microfabricated to define a sample inlet port and a mesoscale flow system, which extends from the inlet port. A polynucleotide polymerization reaction chamber containing reagents for polymerization and amplification of a polynucleotide is in fluid communication with the inlet port. A heat source and, optionally, a cooling source are used to heat and/or cool the chip.

Wilding and co-workers, Nucleic Acids Res., 24:380-385 (1996), demonstrated that PCR could be carried out in a microfabricated silicon glass chip-like chamber. By contacting enclosed 12 microliter reaction chambers microfabricated in glass to a block heater which cycled between two temperatures, they were able to obtain effective and reproducible PCR amplification, as confirmed by removing the PCR product and evaluating it using capillary electrophoresis. Similarly, Northrup and co-workers, Anal. Chem., 68:4081-4086 (1996), accomplished PCR amplification of DNA in a microfabricated silicon PCR device that could be directly interfaced with an electrophoretic chip for PCR product analysis. The device contained disposable polypropylene liners to retain the PCR mixture which could be cycled between two temperatures using polysilicon heaters in direct contact with the PCR chamber and cooled either passively or by air drawn along the heater surfaces of the reaction chamber. The device was interfaced with the electrophoretic chip by forcing it into the 1 mm drilled holes in the electrophoretic chip.

All of the above references, however, describe PCR amplification methods wherein the vessel containing the sample is contacted directly by a heater or another heat source, which transfers heat to the vessel in which the sample is contained. The vessel, in turn, heats the sample. Since these techniques rely on the intimate contact between the circulating medium and the reaction vessel, the surface-to-volume ratio of the reaction vessel is of utmost importance to the effectiveness of the heating step; the higher that ratio the better the PCR reaction.

PCT publication WO 96/41864 discloses a diode laser heated microreaction chamber with a sample detection device. A heat source, such as an IR or UV source, is used to heat the reagents to a thermally induced chemical reaction. Such heating device can be used, for example, in conjunction with the microfabricated reactor described in U.S. Pat. No. 5,639,423.

U.S. Pat. Nos. 6,413,766 and 6,210,882, which are incorporated herein by reference, disclose thermocycling using both a non-contact heating source and a non-contact cooling source. The heating source is provided by optical energy from an IR source. The cooling source is provided by forcing air across the reaction vessel. The temperature sensor in the system, however, is a thermocouple that requires direct contact with the sample fluid.

None of the above references teach methods and systems for performing ultrafast and reliable multiplexed thermocycling using a non-contact heating source for providing sharp and rapid transitions from one temperature to another.

There is a need, therefore, for improved methods and systems for a remote multiplex heating of small samples on a microchip that delivers the heat to multiple chambers simultaneously, either from a single heat source or from multiple heat sources. There is a further need for such methods and apparatus for use with miniaturized thermocylcing, such as that for the polymerase chain reaction (PCR) amplification. Remote heating is used herein to describe temperature measuring without directly contacting the solution of interest.

### SUMMARY OF THE INVENTION

Using a spinning microchip fabricated from glass, silicon, ceramic or plastic, infrared (IR)-mediated temperature cycling of small volumes of solution is possible in multiple chambers on the same device. The present invention approach, which allows for IR heat to be delivered from several low-power IR sources to many microareas (microchannels, microchambers, etc.) on a circular microchip, affords a method and system for multiplexed thermocycling, such as that for PCR-amplification of DNA, on a single microchip device. The IR sources are positioned relative to the microchip in a manner that allows maximum, efficient and equivalent exposure of the IR radiation to the micro-heating areas. By spinning the circular microchip at the appropriate speed, centrifugal forces can be utilized to drive solution from a loading reservoir into the thermocycling chamber where heating occurs for temperature modulation or cycling of the solution. Continued spinning at the appropriate speed allows the radiation from the multiple IR sources to become impingent on all micro-heating areas to avoid heterogenous heating of the microareas. If temperature cycling is involved, air flow over the surface may be exploited to assist in the cooling process, thus accelerating the speed of each cycle and ultimately the overall temperature cycling process. Once heating at the appropriate temperature is complete, accelerated spinning allows for the solution to be forced out of the heating microarea to a recovery reservoir.

In another embodiment, fiber optics are used to direct radiation from a heating source or multiple heating sources directly to the micro-heating areas on a microchip.

Depending on the system used, the microchip can be a spinning chip or an immobile chip having a plurality of micro-heating areas thereon. In the case of the spinning chip, the micro-heating areas are located in a circular configuration on the chip, so the micro-heating areas can be accessed by static heating source(s) by spinning the microchip.

### BRIEF DESCRIPTION OF THE DRAWINGS

FIG. 1 shows a 96-microchamber plate irradiated by two IR sources focused onto a bundle of 48 optical fibers;

FIG. 2 shows a circular 20-chamber microchip with a single IR source;

FIG. 3 shows the details of a micro-heating area designed for exploiting centrifugal force for microfluidics;

FIG. 4 shows a spinning microchip with multiple IR sources positioned below the microchip and remote temperature sensing;

FIG. 5 shows a spinning microchip with a bank or array of IR sources delivering IR radiation to the microchip via optical fibers;

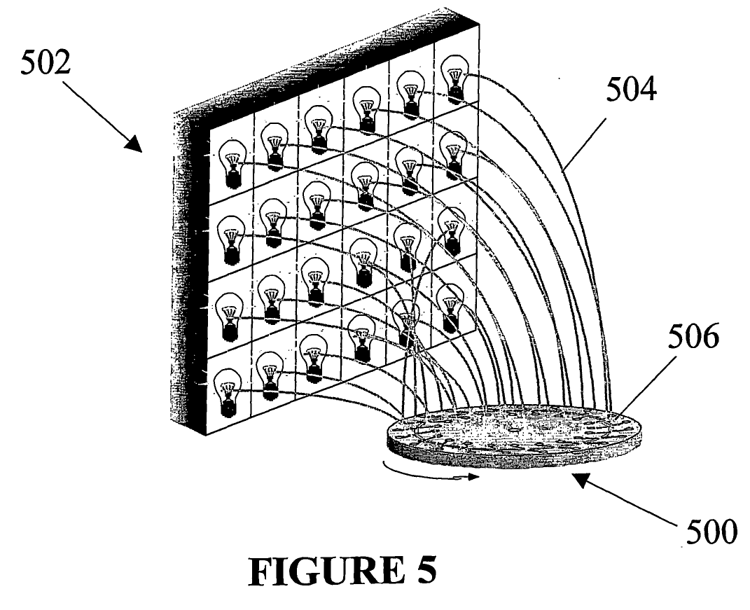

FIG. 6 shows a 3-dimensional view of a spinning PCR microchip with a perimeter IR sources delivering IR radiation to the microchip via waveguides fabricated directly into the chip;

FIG. 7 shows a top view of a spinning PCR microchip with a perimeter IR sources delivering IR radiation to the microchip via waveguides fabricated directly into the chip; and

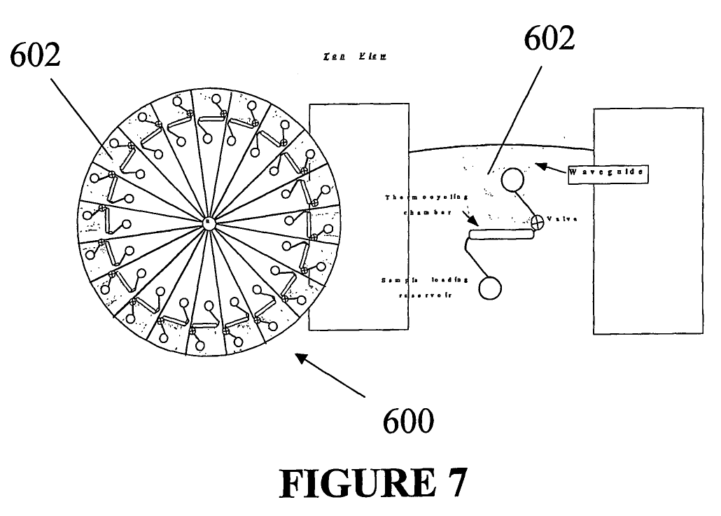

FIG. 8 shows a high throughput 48-chamber microchip capable of accepting and thermocycling 48 samples.

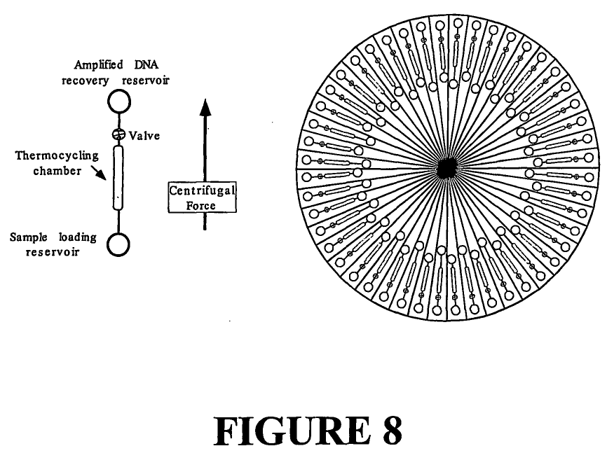

### DETAILED DESCRIPTION OF THE INVENTION

The present invention is generally directed to an apparatus and method for performing remote, rapid, and accurate multiplexed thermocycling of small volume samples on a microchip. Remote heating, cooling, and/or temperature measuring, in the context of this application, is used to describe the process of heating, cooling, and/or temperature measuring without directly contacting the solution of interest. The term “small volume” as used herein refers to volumes in the picoliters (pL) to microliters (μL) range, preferably about 100 pL to about 100 μL, most preferably about 1 nL to about 10 μL.

Applications of the thermocycling method of the present invention are numerous and generally encompass any analytical system in which the temperature of a sample is regulated and/or changed. The present invention is particularly applicable to analytical systems wherein fast or ultrafast transition from one temperature to the next is needed, and in which it is important that exact or nearly exact temperatures be achieved.

For example, the present apparatus and methods are suitable for testing and incubation and treatment of biological samples typically analyzed in a molecular biology laboratory or a clinical diagnostic setting. The accuracy of the thermocycling method of the present invention makes it particularly suitable for use in nucleic acid replication by the polymerase chain reaction (PCR). Any reaction that benefits from precise temperature control, rapid heating and cooling, continuous thermal ramping or other temperature parameters or variations can be accomplished using this method discussed herein. Other applications include, but are not limited to, the activation and acceleration of enzymatic reactions, the deactivation of enzymes, the treatment/incubation of protein-protein complexes, DNA-protein complexes, DNA-DNA complexes and complexes of any of these biomolecules with drugs and/or other organic or inorganic compounds to induce folding/unfolding and the association/dissociation of such complexes. The following applications illustrate the usefulness of the present thermocycling apparatus and methods, representing only some of the possible applications.

A common procedure in the protocols of molecular biology is the deactivation of proteins through heat. One of the most basic procedures in molecular biology is ihe cleavage of proteins and peptides into discrete fragments by proteases/digestion enzymes, such as trypsin. A thermocycling procedure is typically used to activate the enzyme at an elevated temperature followed by: the incubation of the enzyme during the reaction to sustain the enzymatic catalysis; the heat inactivation of the enzyme; and the final treatment/analysis at ambient temperature. Typically, the reaction components are incubated at 40° C. for 60 minutes until the reaction is completed, after which the enzyme activity has to be stopped to avoid unspecific cleavage under uncontrolled conditions. Many enzymes, such as trypsin, can be irreversibly inactivated by incubation for 10 minutes at higher temperature, such as 95° C. The sample is then cooled back to ambient temperature and ready for downstream analysis. Such deactivation of enzymes is taught, for example, in Sequencing of proteins and peptides: Laboratory Techniques in Biochemistry and Molecular Biology, ed. G. Allen, pages 73-105.

The same principle of heat inactivation can be used to inactivate restriction endonucleases that recognize short DNA sequences and cleave double stranded DNA at specific sites within or adjacent to the recognition sequence. Using the appropriate assay conditions (for example, 40° C. for 60 min), the digestion reaction can be completed in the recommended time. The reaction is stopped by incubation of the sample at 65° C. for 10 minutes. Some enzymes may be partially or completely resistant to heat inactivation at 65° C., but they may be inactivated by incubation for 15 minutes at 75° C. Such methods are taught, for example, by Ausubel et al. Short Protocols in Molecular Biology, 3rd Ed., John Wiley & Sons, Inc. (1995) and Molecular Cloning: A Laboratory Manual, J. Sambrook, Eds. E. F. Fritsch, T. Maniatis, 2nd Ed.

Similar to the heat inactivation of proteins for the control of enzymatic activity, the sample processing of proteins for electrophoretic analysis often requires the denaturation of the protein/peptide analyte before the separation by electrophoretic means, such as gel electrophoresis and capillary electrophoresis, takes place. For example, a 5 minute heat denaturation (which provides for the destruction of the tertiary and secondary structure of the protein/peptide) at 95° C. in an aqueous buffer in the presence or absence of denaturing reagents, such as SDS detergent, allows the size dependent separation of proteins and peptides by electrophoretic means. That is taught, for example, in Gel Electrophoresis of Proteins: A Practical Approach, Eds. B. D. Hames and D. Rickwood, page 47, Oxford University Press (1990).

Thermocycling of samples is also used in a number of nonenzymatic processes, such as protein/peptide sequencing by hydrolysis in the presence of acids or bases (for example, 6M HCl at 110° C. for 24 hours) into amino acids. Studies involving the investigation of the interaction of biomolecules with drugs and/or drug candidates are frequently conducted under conditions requiring precise temperature control to obtain binding characteristics, such as kinetic association/dissociation constants.

Those applications for the thermocycling taught by the present invention will find use, for example, as a diagnostic tool in hospitals and laboratories such as for identifying specific genetic characteristics in a sample from a patient, in biotechnology research such as for the development of new drugs, identification of desirable genetic characteristics, etc., in biotechnology industry-wide applications, and in scientific research and development efforts.

Thus, the samples subjected to the thermocycling methods of the present invention will vary depending on the particular application for which the methods are being used. Samples will typically be biological samples, although accurate heating and cooling of non-biological samples is equally within the scope of this invention.

Heating of the sample is accomplished through the use of optical energy from a remote heat source. Preferably, this optical energy is derived from an IR light source which emits light in the wavelengths known to heat water, which is typically in the wavelength range from about 0.775 μm to 7000 μm. For example, the infrared activity absorption bands of sea water are 1.6, 2.1, 3.0, 4.7 and 6.9 μm with an absolute maximum for the absorption coefficient for water at around 3 μm. The IR wavelengths are directed to the vessel containing the sample, and because the vessel is made of a clear or translucent material, the IR waves act directly upon the sample to cause heating of the sample. Although some heating of the sample might be the result of the reaction vessel itself absorbing the irradiation of the IR light, heating of the sample is primarily caused by the direct action of the IR wavelengths on the sample itself.

Typically, the heating source will be an IR source, such as an IR lamp, an IR diode laser or an IR laser. An IR lamp is preferred, as it is inexpensive and easy to use. Preferred IR lamps are halogen lamps and tungsten filament lamps. Halogen and tungsten filament lamps are powerful, and can feed several reactions running in parallel. A tungsten lamp has the advantages of being simple to use and inexpensive, and can almost instantaneously (90% lumen efficiency in 100 msec) reach very high temperatures. A particularly preferred lamp is the CXR, 8V, 50 W tungsten lamp available from General Electric. That lamp is inexpensive and convenient to use, because it typically has all the optics necessary to focus the IR radiation onto the sample; no expensive lens system/optics will typically be required.

In a preferred embodiment, the optical energy is focused on the sample by means of IR transmissible lenses so that the sample is homogeneously irradiated. That technique avoids “hotspots” that could otherwise result in the creation of undesirable temperature differences and/or gradients, or the partial boiling of the sample. The homogeneous treatment of the sample vessel with optical energy therefore contributes to a sharper temperature profile. The homogenous sample irradiation can further be enhanced through the use of a mirror placed on the opposite site of the IR source, such that the reaction vessel is placed between the IR source and the mirror. That arrangement reflects the radiation back onto the sample and substantially reduces thermal gradients in the sample. Alternatively, the radiation can be delivered by optical IR-transparent fiberglass, for example, optical fiberglass made from waterfree quartz glass that is positioned around the reaction vessel and that provides optimal irradiation of the sample.

Heating can be effected in either one step, or numerous steps, depending on the desired application. For example, a particular methodology might require that the sample be heated to a first temperature, maintained at that temperature for a given dwell time, then heated to a higher temperature, and so on. As many heating steps as necessary can be included.

Similarly, cooling to a desired temperature can be effected in one step, or in stepwise reductions with a suitable dwell time at each temperature step. Positive cooling is preferably effected by use of a non-contact air source that forces air at or across the vessel. Preferably, that air source is a compressed air source, although other sources could also be used. It will be understood by those skilled in the art that positive cooling results in a more rapid cooling than simply allowing the vessel to cool to the desired temperature by heat dissipation. Cooling can be accelerated by contacting the reaction vessel with a heat sink comprising a larger surface than the reaction vessel itself; the heat sink is cooled through the non-contact cooling source. The cooling effect can also be more rapid if the air from the non-contact cooling source is at a lower temperature than ambient temperature.

Accordingly, the non-contact cooling source should also be positioned remotely to the sample or reaction vessel, while being close enough to effect the desired level of heat dissipation. Both the heating and cooling sources should be positioned so as to cover the largest possible surface area on the sample vessel. The heating and cooling sources can be alternatively activated to control the temperature of the sample. It will be understood that more than one cooling source can be used.

Positive cooling of the reaction vessel dissipates heat more rapidly than the use of ambient air. The cooling means can be used alone or in conjunction with a heat sink. A particularly preferred cooling source is a compressed air source. Compressed air is directed at the reaction vessel when cooling of the sample is desired through use, for example, of a solenoid valve which regulates the flow of compressed air at or across the sample. The pressure of the air leaving the compressed air source can have a pressure of anywhere between 10 and 60 psi, for example. Higher or lower pressures could also be used. The temperature of the air can be adjusted to achieve the optimum performance in the thermocycling process. Although in most cases compressed air at ambient temperature can create enough of a cooling effect, the use of cooled, compressed air to more quickly cool the sample, or to cool the sample below ambient temperature might be desired in some applications.

A device for monitoring the temperature of the sample, and a device for controlling the heating and cooling of the sample, are also provided. Generally, such monitoring and controlling is accomplished by use of a microprocessor or computer programmed to monitor temperature and regulate or change temperature. An example of such a program is the Labview program (National Instruments, Austin, Tex.). Feedback from a temperature sensing device, such as a thermocouple or a remote temperature sensor, is sent to the computer. In one embodiment, the temperature sensing device provides an electrical input signal to the computer or other controller, which signal corresponds to the temperature of the sample. Preferably, the thermocouple, which can be coated or uncoated, is placed in a temperature sensing reaction vessel placed adjacent to the reaction vessel containing the sample to be tested. The temperature sensing reaction vessel should be of the same type as the sample containing reaction vessel, only containing a blank, such as water or a buffer solution instead of sample. Alternatively, the thermocouple can be placed directly into the sample vessel, provided that the thermocouple does not interfere with the particular reaction or affect the thermocycling, and provided that the thermocouple used does not act as a heat sink. A suitable thermocouple for use with the present invention is constantan-copper thermocouple. In some instances it might be an advantage to sense the sample temperature through a thermosensor directly measuring the reaction vessel, or the sample itself.

In a most preferred embodiment, temperature is monitored and controlled through a remote temperature sensing means. For example, a thermo-optical sensing device can be placed above an open reaction vessel containing the sample being thermocycled. Such a device can sense the temperature on a surface, here the surface of the sample, when positioned remotely from the sample.

Remote sensing of the temperature of a solution within a small volume chamber can be accomplished by measuring changes in the refractive index of the solution. Optical interferometric sensing, preferably Extrinsic Fabry-Perot Interferometry (EFPI), technology is capable of measuring very small distances based on the formation of a low-finesse Fabry-Perot cavity between two reflective surfaces. That is accomplished by passing light through an optical fiber, and measuring differences in the light reflected from the two reflective surfaces back through the same fiber. Often, one of those interfaces is the fiber/air interface at the polished end of the fiber, but in microchip measurements, the top surface of the device, and the bottom of the microfabricated chamber can be used to define at least one cavity. Constructive and destructive interference occurs between the reflected light waves based on the path length difference traversed. Within the microchip, the distance of the light path through the solution, to reflect from the bottom of the microfabricated chamber, changes as the refractive index of the solution changes. Since the refractive index is a function of the temperature of the solution, that change in the distance traveled by the light reflected from the chamber bottom can be used to determine the temperature of a solution within the microchip chamber. With a fiber placed above the section of the microchip to be interrogated, and with the appropriate calibration, the solution temperature can be determined rapidly (in microseconds) and with an accuracy that is on the order of about ±0.5° C. In some applications, multiple reflections may be possible. In those cases, the individual path lengths can be isolated using optical path length multiplexing methods.

Signals from the computer, in turn, control and regulate the heating and cooling means, such as through one or more switches and/or valves. The desired temperature profile, including dwell times, is programmed into the computer, which is operatively associated with heating and cooling means so as to control heating and cooling of the sample based upon feedback from the temperature sensor and the predetermined temperature profile.

Accordingly, the methods of the present invention provide for the use of virtually any temperature profile/dwell time necessary. For example, cleavage of proteins through use of proteases or digestion enzymes might require use of different temperatures, each of which must be precisely maintained for various amounts of time. Activation of restriction endonucleases might similarly require achieving and maintaining two or three different temperatures. Protein or peptide sequencing can require the steady maintenance of a high temperature for an extended period of time.

The above apparatus provide for rapid heating and cooling of a sample in a precise and easy to replicate manner. Heating can be effected for example as quickly as 10° C. per second when using approximately 15 to 50 μL volumes of sample in a microchamber and as rapidly as 100° C. per second when using nL volume samples in a capillary. Cooling can be effected quickly, typically in the range of between about 5 and 50° C. per second. The increased effectiveness of heating and cooling improves the cycling process and sharpens the temperature profile. This means that the desired reaction can be conducted under more optimal thermal conditions than in conventional instruments. Thermal gradients in the reaction medium frequently observed in instrumentation using a contact heat source are detrimental to the specificity of the reaction. Those thermal gradients are substantially reduced in the IR mediated heating, particularly when the heat source is strong enough to penetrate the aqueous mixture and provide sufficient irradiation to the opposite side of the reaction vessel. Non-contact, remote rapid cooling, heating, and temperature sensing, such as that provided in the present invention, also contributes to the ability to obtain sharp transition temperatures in minimum time and to achieve fast and accurate temperature profiles.

Translating IR-mediated thermocycling to a multiplexed system requires that radiation from an IR source(s) be delivered to a plurality of heating micro-areas on a microchip. That can be accomplished by a number of embodiments.

A first embodiment involves using a fiber optic bundle to deliver heating radiation to the micro-heating areas. The irradiation from a powerful IR source can be focused into a bundle of optical fibers having the appropriate character for propagating the IR from the source to the micro-heating area In order for that to be a functional approach: 1) the IR source would have to have enough power to provide the appropriate amount of power to each micro-heating area; 2) there would have to be equivalent power distribution to each chamber; 3) each fiber would have to have equivalent radiation transmission properties and minimal power loss over the length of the fiber; and 4) there would have to be cooling homogeneity over the entire surface of the chip. Optical fibers produced for the telecommunications industry should be ideal for this purpose since they are designed for light propagation in the 1.3-1.4 μm range, exactly the preferred part of the spectrum used in IR-mediated heating here. In addition, in light of the rigorous regulations and quality control associated with optical fiber manufacturing, it is not unreasonable to expect that the requirements detailed above could be met with such optical fibers.

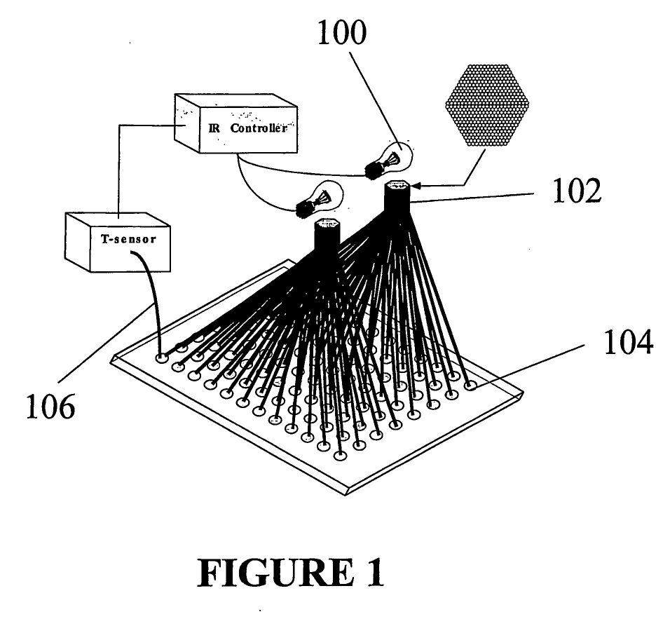

FIG. 1 shows a schematic of a system for the IR heating 96 micro-heating areas 104 using two IR sources 102, where each feeds power into a bundle of 48 fibers 102. The power distribution may come from as few as one IR source, with the energy focused into a bundle of 96 fibers, or a plurality of IR sources with each focused onto the appropriate number of fibers. For example, if four IR sources are used, each source would focus onto 24 fibers; if eight IR sources are used, each source would focus onto 12 fibers; etc.

With this configuration, one of the micro-heating areas 104 would be interrogated by a remote temperature-sensing device 106. That can be an IR pyrometer (equivalent technology to that used on ear-based temperature sensors) which senses the temperature at the chip surface. That requires the calibration of sample temperature with that on the surface. Alternatively, and more preferably, a refractive index (RI) detector, such as an interferometer, can be used which would sense the solution temperature directly.

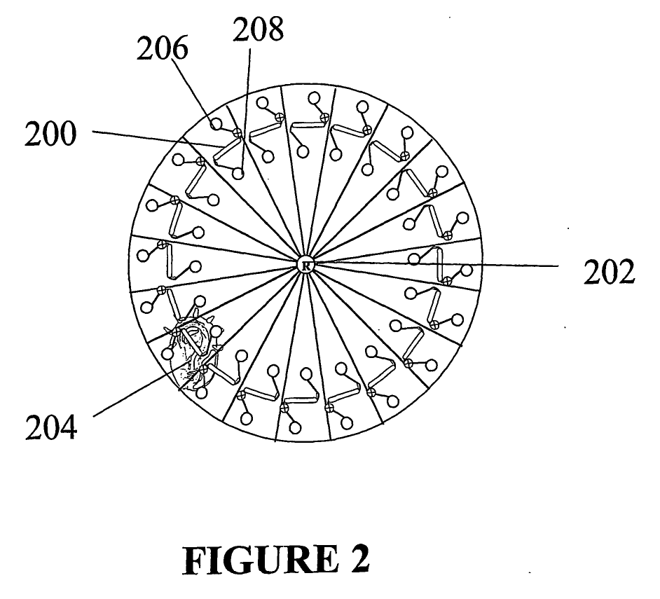

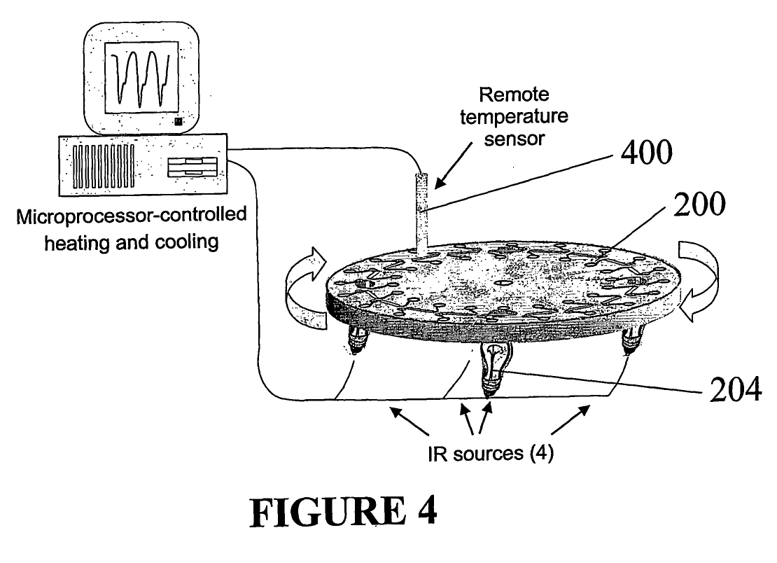

A second embodiment involves a spinning microchip having a thermocycling chamber/reservoirs design. In this approach, all IR sources are impingent on all micro-heating areas. That can be accommodated by using a circular microchip depicted in FIG. 2, where the thermocycling chambers 200 are arranged in a circular configuration on the chip. With that approach, all of the micro-heating areas fall on a concentric ring equidistant from the rotor 202, so that a spinning about the rotor would allow for all micro-heating areas to be accessed from a single static point. That creates the opportunity for an IR source(s) 204 to irradiate all micro-heating areas while the chip spins, thus avoiding inconsistencies with power impingent on any particular micro-heating area In addition, remote temperature sensing (not shown in FIG. 2, shown as element 400 in FIG. 4) would allow for more than a single micro-heating area to be interrogated for solution temperature.

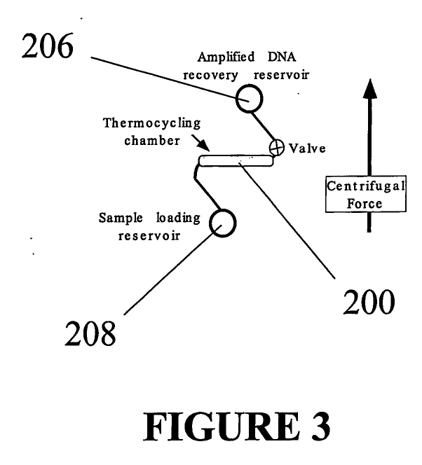

With a microchip containing 20 thermocycling chambers 200, it is possible to have the stationary light source 204 positioned below or above the microchip so that all 20 chambers are irradiated as the microchip spins at some defined speed. Moreover, with a chamber-reservoir configuration as shown in FIGS. 2 and 3, centrifugal force can be exploited to move the reaction mixture from the loading reservoir 208 into the thermocycling chamber 200 and eventually into the recovery reservoir 206.

One of the advantages of the spinning microchip design, but not limited thereto, is that should there be power limitations with a single IR source, multiple sources could be arranged to impact the microchip simultaneously as diagrammed in FIG. 4.

With multiple IR sources 204, whereby the number that could be used is simply limited by the size of the IR lamp, heating is enhanced by the delivery of adequate power to each thermocycling chamber. With that configuration, the power experienced by each chamber is an average of that delivered by each lamp; therefore, slight differences that may exist in the power output from each lamp are of little consequence.

A third embodiment involves an IR bank and optical fibers to deliver IR radiation to micro-heating areas. This embodiment marries the concept of the first two embodiments into a single concept. With this configuration, there is a bank or array of IR sources 502 remote from the chip and, using optical fibers 504, the IR light is brought to the micro-heating areas 506 without over crowding the space above or below the chip.

Spinning the microchip 500 at a speed that allows for delivery of the appropriate amount of power to each micro-heating area 506 should allow for the desired temperature acquisition. In addition, a remote temperature probe (not shown) position above the micro-heating area 506 of the spinning chip 500 allows for temperature interrogation of all of the micro-heating areas, provided the time constant for sensing is small enough.

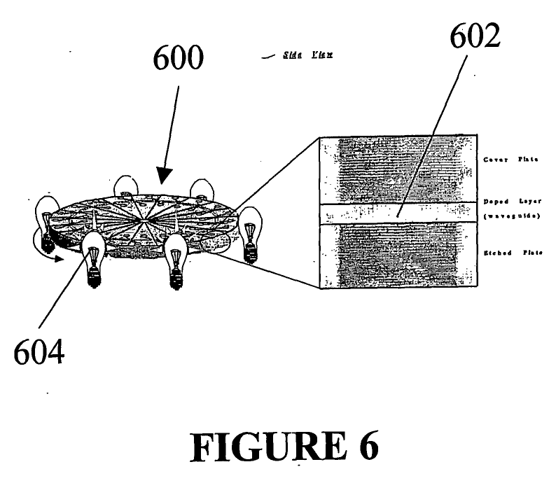

A fourth embodiment involves the used of a waveguide microfabricated into the microchip to deliver radiation from IR sources, placed at the perimeter of the spinning microchip, to the micro-heating areas. That embodiment is illustrated in FIG. 6. The microchip 600 contains a middle layer 602 that is doped with the appropriate material, such as silicon dioxide, to create a waveguide that allows direct propagation of IR light from a source(s) 604 located on the perimeter of the microchip 600 through the chip to the micro-heating areas.

An alternative approach for directing IR light to the microchip is to use microminiatured IR sources that is built into the stage that accommodates the chip. The fabrication of micro-IR sources 1 mm in size and capable of generating about 1 watt of IR power has been demonstrated by Corman et al. (J. Microelectromechanical Systems, 9:509-516, 2000). Using a silicon-based platform containing the desired (necessary) number of IR sources, a concentric ring of microfabricated IR sources can provide adequate power to reach and maintain the desired temperatures for thermocycling.

Although the previous Figures show a twenty micro-heating areas spinning microchip, any number of micro-heating areas can be designed incorporating the same elements as those of the previous Figures for each micro-heating area, including the sample loading reservoir, the thermocycling chamber, and the recovery reservoir. FIG. 6 shows an example of a 48-chamber microchip capable of multiplexing 48 simultaneous thermocycling reactions.

Although the Figures show microchips having only a single ring of micro-heating areas, the microchip can also have several concentric rings of micro-heating areas. Each concentric ring can be heated by one or several heating sources. In another embodiment, a single source can heat the several concentric rings by having a fiber optic bundle directing the heating radiation to each of the concentric ring.

Because the microchip of the present invention spins in circular motion, the centrifugal forces generated during rotation can be exploited to move the solution from the loading reservoir into the thermocycling chamber and eventually into the recovery reservoir. Examples in the literature have shown the use of centrifugal for such purposes. Duffy et al. (Anal. Chem., 71: 4669-4678, 1999) demonstrate this with the mixing of the appropriate reagents with a sample in a homogenous enzyme assay. The present invention uses centrifugal forces to load sample, wash material and elute DNA from an appropriately-design microchip, similar to the teaching of Duffy et al. The required centrifugal force can be calculated from the average velocity of the liquid (U) and its volumetric flow rate (O) depend on the Theological properties of the liquid, the size, location, and configuration of the channels, and the rate of rotation, through equations 1 and 2
U=d_{H}²ρω²RΔr/32ηL (1)
Q=UA=Ad_{H}²ρω²RΔr/32ηL (2)
where ρ and η are the density and viscosity of the liquid, respectively, A is the cross-sectional area of the channel; d_{H} is the hydraulic diameter of the channel (defined as 4A/P, where P is the perimeter of the channel); L is the length of the channel; ω is the angular velocity; R is the average distance of the liquid in the channels from the center of the disk; and r is the radial extent of the fluid subject to centrifugal force. Using these equations and the like, the rotational speed and the size of the device needed to create adequate pressure to drive fluid through empty and packed microchannels can be determined.

It is further possible to exploit the spinning motion to direct enhanced air flow over the surface of the chip. That is important during the cooling part of the thermocycling and optimizing the design of the instrument cover would allow for optimal use of that air flow to cool the surface when needed.

The solution can be retained in the thermocycling chamber by a valve that will only be activated after the thermocycling is complete. Actuation of that valve then allows for the solution to enter into the recovery reservoir after the end of the thermocycling process.

1. A system for multiplexed thermocycling, comprising:
a microchip having a plurality of micro-heating areas thereon; and
a non-contact heating source for the micro-heating areas.

2. The system of claim 1, further comprising non-contact means for cooling the micro-heating areas.

3. The system of claim 2, wherein the means for cooling is a compressed air source.

4. The system of claim 3, wherein the compressed air source has means for chilling air.

5. The system of claim 2, wherein the air from said cooling means has a pressure of between about 1 and 150 psi.

6. The system of claim 2, wherein the rate of flow of air from said compressed air source is controlled by a solenoid valve.

7. The apparatus of claim 2, wherein said cooling means is structured to cause forced air to impinge on the micro-heating areas from a position angularly offset with respect to the direction of heat applied from said heating means.

8. The system of claim 1, further comprising means for monitoring the temperature of the micro-heating areas.

9. The system of claim 8, wherein the means for monitoring the temperature is selected from the group consisting of a thermocouple and a remote temperature sensor.

10. The system of claim 9, wherein the remote temperature sensor is an interferometer.

11. The system of claim 10, wherein the interferometer is and Extrinsic Fabry-Perot Interferometer.

12. The system of claim 1, further comprising a microprocessor operatively associated with the heating means, the cooling mean, the temperature monitoring means, and the microchip.

13. The system of claim 12, wherein said microprocessor means has means for establishing a plurality of desired temperatures and a plurality of desired dwell times at each desired temperature.

14. The system of claim 12, wherein said microprocessor means has means for effecting DNA amplification in a sample.

15. The system of claim 1, wherein the non-contact heating source is at least one IR source.

16. The system of claim 15, wherein the IR source is a halogen lamp.

17. The system of claim 15, wherein the IR source is a tungsten lamp.

18. The system of claim 15, wherein said IR source is disposed in a spaced relationship with respect to the microchip.

19. The system of claim 15, further comprising filter means interposed between the IR source and the microchip.

20. The system of claim 1, further comprising a fiber optic bundle for directing radiation from the non-contact heating source to each of the micro-heating areas.

21. The system of claim 1, wherein the micro-heating areas comprises a sample loading reservoir, a thermocycling chamber, and a recovery reservoir fluidly connected with each other.

22. The system of claim 1, wherein the thermocycling chamber and the recovery reservoir is fluidly connected through a valve.

23. The system of claim 1, wherein the micro-heating areas are arranged in a circular ring on the microchip.

24. The system of claim 1, wherein the chip is mounted on a rotor that is capable of spinning the chip around its center.

25. The system of claim 24, wherein the micro-heating areas are arranged in a circular ring on the microchip equidistant from the center.

26. The system of claim 1, wherein the microchip includes a waveguide doped therein to conduct radiation from the non-contact heating source to the micro-heating areas.

27. A method for multiplexed thermocycling, comprising the steps of:
a) providing a microchip having a plurality of micro-heating areas thereon;
b) providing a small volume sample in each of the micro-heating areas;
c) heating the samples using non-contact heating source;
d) cooling the sample using non-contact means for cooling; and
e) repeating steps c) and d) to perform a desired number of cycles.

28. The method of claim 27, wherein the non-contact heating source is at least one IR source.

29. The method of claim 28, wherein the IR source is a halogen lamp.

30. The method of claim 28 wherein the IR source is a tungsten lamp.

31. The method of claim 28, wherein said IR source is disposed in a spaced relationship with respect to the microchip.

32. The method of claim 27, wherein the heating step comprises conducting radiation from the non-contact heating source through a fiber optic bundle which conducts the radiation from the non-contact heating source to each of the micro-heating areas.

33. The method of claim 27, wherein the micro-heating areas comprises a sample loading reservoir, a thermocycling chamber, and a recovery reservoir fluidly connected with each other.

34. The method of claim 33, wherein the thermocycling chamber and the recovery reservoir is fluidly connected through a valve.

35. The method of claim 27, wherein the micro-heating areas are arranged in a circular ring on the microchip.

36. The method of claim 27, wherein the microchip is mounted on a rotor that is capable of spinning the chip around its center.

37. The method of claim 27, wherein the micro-heating areas are arranged in a circular ring on the microchip equidistant from the center.

38. The method of claim 27, wherein the heating step comprises spinning the micro chip such that radiation from at least one stationary heating source impinges on the micro-heating areas as they passes the at least one stationary heating source.
